(19) United States
(12) Patent Application Publication
(10) Pub. No.: US 2025/0248944 A1
(43) Pub. Date: Aug. 7, 2025

(54) SOFTGEL CAPSULES HAVING A FILL COMPOSITION COMPRISING APPLE CIDER VINEGAR

(71) Applicant: CAPTEK SOFTGEL INTERNATIONAL, INC., Cerritos, CA (US)

(72) Inventors: Diana Yoonchung Paik LEE, Fullerton, CA (US); Hong Anh Nguyen, Westminster, CA (US); Timothy Brian Chiprich, Huntington Beach, CA (US)

(73) Assignee: CAPTEK SOFTGEL INTERNATIONAL, INC., Cerritos, CA (US)

(21) Appl. No.: 18/429,843

(22) Filed: Feb. 1, 2024

(51) Int. Cl. A61K 9/48; A61K 36/73; A61K 45/06

(52) U.S. Cl. CPC A61K 9/4825; A61K 9/4858; A61K 36/73; A61K 45/06

(57) ABSTRACT

Softgel capsules have a liquid fill composition of 30% to 40% wt/wt of an edible oil or edible fat; 0.2% to 30% wt/wt of a mineral acetate powder, with the proviso that the mineral acetate powder does not comprise disodium acetate; 1% to 8% wt/wt of one or a combination of a suspending agent and/or a wetting agent; and apple cider vinegar powder as a balance of the liquid fill material encapsulated in an outer softgel shell.

### TECHNICAL FIELD

The present disclosure relates generally to the field of softgel capsules, and more particularly to softgel capsules having a fill material that includes apple cider vinegar.

### BACKGROUND

Apple cider vinegar (“ACV”) is derived from fermentation of sweet apples and is a weak acid (acetic acid). For centuries, ACV has been used and treasured in age-old wellness practices. Apple cider vinegar supplements have become increasingly popular as a way to lower sugar cravings and decrease cholesterol levels. Supplements are a convenient way of consuming ACV without the harsh taste that can accompany a liquid formula. When adding ACV to a daily routine, it is important to find a supplement with a high enough concentration to reap the desired benefits. Not all ACV supplements contain enough acetic acid, and some have a high amount of sugar. A minimal amount of ACV is 200 mg.

Some studies have suggested that ACV can lower blood sugar, which may be beneficial for someone with diabetes, can calm acid reflux, and can aid in weight loss. Today, ACV dietary supplements have become popular with adults following healthy diet and weight management routines.

Supplements containing ACV are commercially available as solid capsules, hardshell capsules filled with a solid, or as gummies. It is difficult to put ACV in liquid form in a softgel in any meaningful dosage because the acetic acid deteriorates the softgel and ultimately causes the softgel to leak. Gelatin is particularly susceptible to ACV. Applicant's prior attempts to make softgels failed because the softgels developed leaks over time.

The Bragg brand of apple cider vinegar hardshell capsules include sodium diacetate. Sodium diacetate was tested in softgel liquid fill materials by Applicant, and it was determined that sodium diacetate causes holes to form in the softgel shell.

There is a need for a softgel enclosing a liquid fill material that includes ACV to provide 750 mg of total acetic acid per serving, in particular in a softgel that is shelf stable, i.e., softgels that do not leak acetic acid over time.

### SUMMARY

In one aspect, softgel capsules are disclosed herein that have a liquid fill composition comprising: 30% to 40% wt/wt of an edible oil or edible fat; 0.2% to 30% wt/wt of a mineral acetate powder, with the proviso that the mineral acetate powder does not comprise disodium acetate; 1% to 8% wt/wt of one or a combination of a suspending agent and/or a wetting agent; and apple cider vinegar powder as a balance of the liquid fill material; and an outer softgel shell encapsulating the liquid fill material. In one embodiment or all embodiments, the mineral acetate powder is a blend of calcium acetate powder and zinc acetate powder. The zinc acetate powder is 3% to 6% wt/wt of the blend of calcium acetate powder and zinc acetate powder or 4% to 5% wt/wt of the blend of calcium acetate powder and zinc acetate powder.

In any one of the embodiments or all embodiments, the liquid fill material can include a second active agent, which can be present as 0.01% to 10% wt/wt thereof. The second active agent is selected from the group consisting of oil soluble vitamins, water soluble vitamins, minerals, herbal extracts, amino acids, and combinations thereof.

In any one of the embodiments or all embodiments, a suspending agent and a wetting agent can be present in the liquid fill material. In one embodiment, the suspending agent and wetting agent are present in a wt/wt ratio of 1:1. The suspending agent is selected from the group consisting of beeswax, candelilla wax, carnauba wax, colloidal silicone dioxide, hydrophobic colloidal silica, hydrogenated vegetable oils, glycerol esters of fatty acids with low HLB values, polyvinylpyrrolidone, and mixtures of mono-, di-, and triglycerides/mono- and di-fatty acid esters of polyethylene glycols, gum Arabic, propylene glycol, alginate, Vitamin E polyethylene glycol succinate, and combinations thereof. The wetting agent can comprise lecithin.

In any one of the embodiments or all embodiments, the liquid fill composition comprises the edible oil selected from the group consisting of coconut oil, palm kernel oil, palm oil, soybean oil, safflower oil, cotton seed oil, rapeseed oil, poppy seed oil, corn oil, sunflower oil, olive oil, algae oil, fish oil, fish liver oil, seal oil, krill oil, squid oil, vegetable oil, and combinations thereof. In other embodiments, the liquid fill composition comprises the edible fat, which is selected from the group consisting of lard, tallow fat, and combinations thereof.

In some embodiments, the outer softgel shell has a composition comprising gelatin and a plasticizer. In other embodiment, the outer softgel shell has a composition comprising a modified starch, carrageenan, and a plasticizer.

In one embodiment, the fill composition comprises: 33% to 37% wt/wt of the edible oil or edible fat; 0.4% to 18% wt/wt of the mineral acetate powder; and 1% to 5% wt/wt of the combination of the suspending agent and the wetting agent. The outer softgel shell has a composition that comprises gelatin and a plasticizer or a modified starch, carrageenan, and a plasticizer. In some embodiments or all embodiments, the liquid fill material can have a second active agent, which can be present as 0.01% to 10% wt/wt thereof. In one embodiment or all embodiments, the mineral acetate powder is a blend of calcium acetate powder and zinc acetate powder, where the zinc acetate is 3% to 6% wt/wt or 4% to 5% wt/wt of the blend of calcium acetate powder and zinc acetate powder.

### BRIEF DESCRIPTION OF THE DRAWINGS

FIG. 1 is a two-piece hardshell capsule.

FIG. 2 is a softgel capsule containing a liquid fill material comprising apple cider vinegar.

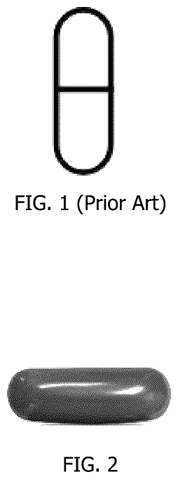

### DETAILED DESCRIPTION

All numerical values set forth herein can be modified by the word “about” whether expressly stated or not. “About” as used herein means plus or minus 5% of a numerical value, or more preferably plus or minus 2%. For ranges, the minimum and maximum values are included in the range and all values therebetween. It is further understood that the endpoints of each range are significant both in relation to the other endpoint, and independently of the other endpoint. Percentages for concentrations are typically % by weight/weight unless expressly stated otherwise. “Substantially free” means 0.01% wt/wt or less are present of the identified substance.

The following description and drawings are illustrative and are not to be construed as limiting. Numerous specific details are described to provide a thorough understanding of the disclosure. However, in certain instances, well-known or conventional details are not described to avoid obscuring the description. Reference to an “embodiment” or “example embodiment” means that a particular feature, structure, or characteristic described in connection with the embodiment is included in at least one embodiment of the disclosure.

Provided herein are softgel capsules enclosing inside of an outer softgel shell a liquid fill material that includes:
30% to 40% wt/wt of an edible oil or edible fat;
0.2% to 30% wt/wt of a mineral acetate powder, with the proviso that the mineral acetate powder does not comprise sodium diacetate;
1% to 8% wt/wt of one or a combination of a suspending agent and a wetting agent; and
apple cider vinegar powder as a balance of the liquid fill material; and an outer softgel shell encapsulating the liquid fill material. More preferably, the liquid fill material includes 33% to 37% wt/wt of an edible oil or edible fat; 0.2% to 30% of mineral acetate powders (free of sodium diacetate); 1% to 5% wt/wt of one or the combination of the suspending agent and the wetting agent; 0.01% to 5% of a stabilizing agent such as a chelating agent and/or an antioxidant; and apple cider vinegar powder as the balance of the liquid fill material. Chelating agents and/or antioxidants can be be added to enhance the stability and shelf life of the first active agent (i.e., the apple cider vinegar) in the final product.

The goal is to provide shelf stable softgel capsules that do not leak acetic acid and can provide a consumer a 750 mg dosage of the acetic acid. The dosage maybe consumed by taking one, two, three, four or five softgel capsules depending upon the size of the softgel capsules. In some embodiments, the serving size is four softgels, i.e., each softgel has a total acetic acid dosage of 187.5 mg. In another embodiment, the serving size is three softgels, i.e., each softgel has a total acetic acid dosage of 250 mg.

Apple cider vinegar (acetic acid) is the primary active in the softgel capsules described herein. The liquid fill material can also include a second active agent. In one aspect, the second active agent is selected from the group consisting of oil soluble vitamins, water soluble vitamins, minerals, herbal extracts, amino acids, and combinations thereof. The softgel capsules can have 0.01% to 10% wt/wt of the second active agent as part of the liquid fill material.

The mineral acetate can be any one of the following acetates individually or in a blend with any number of the acetates: calcium acetate, chromium (II or III) acetate, copper (I or II) acetate, iron (II or III) acetate, magnesium (II or III) acetate, manganese acetate, and zinc acetate. The mineral acetate is present in powder form and is suspended in the edible oil or edible fat. The mineral acetate powder is present as 0.2% to 30% wt/wt, preferably 0.2% to 20% wt/wt, more preferably 1% to 20% wt/wt, and even more preferably 10% to 20% wt/wt of the liquid fill material. In one embodiment, the mineral acetate powder is present as about 15% wt/wt to about 18% wt/wt of the liquid fill material. In one embodiment, the liquid fill material comprises only calcium acetate as 0.2% to 25% wt/wt of the fill material. In another embodiment, the liquid fill material comprises only zinc acetate as 0.2% to 25% wt/wt of the liquid fill material. In another embodiment, the liquid fill material comprises a blend of calcium acetate and zinc acetate.

Various sources of acetic acid were tested in the laboratory to determine their compatibility with the gelatin and plasticizer matrix. Sodium diacetate exhibited the most significant reaction, corroding a hole in the gelatin and causing leakage of the fill material. Lysine acetate powder completely liquefied upon testing. Calcium acetate, when tested alone, partially gelled up and liquified. Zinc acetate showed the least reaction when tested individually. Based on these results, the softgel capsules are free of sodium diacetate, i.e., it is not present in the liquid fill material. Surprisingly, other mineral acetates do not create holes in the softgel shell. Additionally, the combination of apple cider vinegar, calcium acetate, and various zinc sources (such as zinc oxide and zinc gluconate) worked, but not as effectively as desired for a shelf stable product. Surprisingly, the combination of apple cider vinegar, calcium acetate, and zinc acetate exhibited the least reaction with the softgel capsule material, thereby resulting in a shelf stable product.

In one embodiment, the mineral acetate powder is a blend of calcium acetate powder and zinc acetate powder. The zinc acetate powder can be present as 3% to 6% wt/wt of the blend of calcium acetate powder and zinc acetate powder, more preferably 4% to 5% wt/wt of the blend of calcium acetate powder and zinc acetate powder, and even more preferably 4.2% to 4.8% wt/wt of the blend of calcium acetate powder and zinc acetate powder.

The outer softgel shell can be composed of either animal-derived ingredients or vegetarian-sourced ingredients, comprising: 10-30% gelling agent; 2-20% shell plasticizers; 1-5% of shell opacifier, colorants, flavors, and combinations thereof. Some non-limiting examples of gelling agents include hydrocolloids such as gelatin, starch, carrageenan, agar, pectin, xanthan gum, and combinations thereof. Some non-limiting examples of plasticizers include sugar alcohols such as glycerin, sorbitol, maltitol, xylitol, mannitol, erythritol, and combinations thereof.

As used herein, a “softgel capsule” refers to a pharmaceutical device having a shell formed of highly plasticized soft elastic shell material to define at least one chamber for a dosage of fill material. The fill material can be a liquid, suspended powder in a liquid, or a semisolid. The softgel capsule can have a seam or can be seamless, depending upon the method of manufacturing. Seamed softgel capsules can be made using a rotary die encapsulation machine. Seamless softgel capsules can be made by either coacervation or by the “drop method” using concentric nozzles (i.e., a coextrusion process without mechanical shaping). The shell material can be animal gelatin plasticized with a sugar alcohol (e.g., glycerol, sorbitol, maltitol, etc.) or one or more vegetarian capsule-forming materials such as starches, modified starches, carrageenan and alginates or similar polymers, with or without polyhydric alcohol plasticizers. Any softgel shell material is suitable herein. Examples of softgel shells are described in U.S. Pat. Nos. 5,614,217; 7,807,194; U.S. patent publication US 2005/0152969; and U.S. patent publication US 2019/0269623, the contents of each of which is hereby incorporated by reference in their entirety.

Softgel capsules can provide distinct advantages over more traditional dosage forms such as tablets, hard-shell capsules, and liquids. These advantages include patient compliance and consumer preference, improved bioavailability, speed of product development in many cases, shortened manufacturing time, enhanced drug stability due to less exposure of the active ingredient to oxygen, dose uniformity, and product differentiation, for example through novel shapes.

### The Liquid Fill Material Ingredients

The apple cider vinegar can be pasteurized or raw.

Some non-limiting examples of animal and plant edible oils and edible fats include, optionally fractions of, coconut oil, palm kernel oil, palm oil, marine oils (fish oil, fish liver oil, seal oil, krill oil, squid oil, single cell oils from marine and freshwater algae), lard, tallow fat, soybean oil, safflower oil, cotton seed oil, rapeseed oil, poppy seed oil, corn oil, sunflower oil, olive oil, algae oil, vegetable oil, and blends thereof. Hydrogenation may be used to alter the degree of unsaturation of the fatty acids and as such to alter the fatty acid composition.

Some non-limiting examples of suspending agents include: beeswax, candelilla wax, carnauba wax, colloidal silicone dioxide, hydrophobic colloidal silica, hydrogenated vegetable oils, glycerol esters of fatty acids with low HLB values, polyvinylpyrrolidone, and mixtures of mono-, di-, and triglycerides/mono- and di-fatty acid esters of polyethylene glycols, gum Arabic, propylene glycol, alginate, Vitamin E polyethylene glycol succinate (TPGS) (also known as tocofersolan), and combinations thereof.

Some non-limiting examples of wetting agents and emulsifiers include lecithin. Some non-limiting examples of surfactants include polysorbate, phospholipids, sodium lauryl sulfate, monolaurate (Polysorbate 20), Polysorbate 80, cetyltrimethylammonium bromide (CTAB), polyoxyl castor oil (Cremophor EL), hexadecyltrimethylammonium, bromide (HTAB), polyethylene glycol tert-octylphenyl ether (Triton), nonylphenol ethoxylate (Tergitol), and combinations thereof.

In one embodiment, both a suspending agent and a wetting agent are present. The wetting agent can be 0.5% to 25% wt/wt of the oils in the formula. The suspending agent can be 0.5% to 25% wt/wt of the entire fill material.

The softgel capsules disclosed herein can be made with non-GMO and gluten free ingredient. Moreover, there can be no sugars, no artificial colors, no artificial flavors, no artificial sweetener, no chloroform, no garlic, and no preservatives in the fill material composition or the capsule shell composition. Additionally, the softgel capsules are free of sodium diacetate and are optionally free of zinc oxide.

The invention will now be described in detail by way of reference to the following examples. It is to be understood that one, some, or all properties of the various embodiments and examples described herein may be combined to form other embodiments.

### Example 1

TABLE 1
Apple Cider Vinegar - capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | calcium acetate & zinc acetate | 0.2 | 17 | 30 |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

The capsule fill composition can be the fill for vegetarian softgel capsules (typically containing carrageenan) or animal softgel capsules (typically containing gelatin). Two non-limiting example softgel capsule shell materials are provided below.

TABLE 2
Example animal-based softgel capsule shell material

| | | | Example | |
|---|---|---|---|---|
| | | Minimum | approx. | Maximum |
| Ingredient | Example | % wt/wt | % wt/wt | % wt/wt |
| Gelling agent | gelatin | 10 | 17.6 | 20 |
| Plasticizer | glycerin | 1 | 3.5 | 5 |
| Plasticizer | sorbitol | 1 | 3.5 | 5 |
| Water | De-ionized water | 1 | 2 | 10 |
| Colorant | | 0.2 | 0.7 | 2 |

TABLE 3
Example vegetarian softgel capsule shell material

| | | | Example | |
|---|---|---|---|---|
| | | Minimum | approx. | Maximum |
| Ingredient | Example | % wt/wt | % wt/wt | % wt/wt |
| Gelling agent | Modified starch | 5 | 10.5 | 20 |
| Gelling agent | Carrageenan | 1 | 5 | 10 |
| Plasticizer | glycerin | 1 | 4.6 | 10 |
| Plasticizer | sorbitol | 1 | 4.5 | 10 |
| Water | De-ionized water | 1 | 2.8 | 10 |
| Colorant | | 0.2 | 0.8 | 2 |

One example method of making the softgel capsule is a rotary die process. This process is also known as a die roll process because it uses two die rolls facing one another. Each dies roll has a plurality of pockets arranged in longitudinal rows, which each define one half of a softgel capsule shape. Two capsule shell sheets, made from an animal-based shell material or a vegetarian shell material, are fed between the two die rolls from the top (one from the left and one from the right). The liquid fill material according to table 1 is injected between the rolls, between the two capsule shell sheets, into a pocket defined by the two die rolls and is encapsulated within a softgel capsule formed by pressure and heat as the two die rolls roll and meet. This method is described in more detail in Chapter 34 of Aulton's Pharmaceutics: The Design and Manufacture of Medicines, 2017.

### Bottle Stability Testing

45 softgel capsules were placed into a white HDPE bottle, sealed with heat induction, and stored under two different storage conditions: (1) 25° C./60% relative humidity (RH) (real-time), and (2) 40° C./75% RH (accelerated) for stability for each of softgel capsule shell materials discussed above.

TABLE 4
Stability of Apple Cider Vinegar in Animal Gelatin Softgel

| | Acetic Acid Test Results | | | | | |
|---|---|---|---|---|---|---|
| Time Point | Initial | 1 M | 2 M | 3 M | 6 M | 9 M |
| Real Time (25° C./60% RH) | 106% | NA | NA | 100% | 107% | 100% |
| Accelerated (40° C./75% RH) | | 100% | 100% | 100% | 107% | NA |

TABLE 5
Stability of Apple Cider Vinegar in Vegetarian Softgel

| | Acetic Acid Test Results | | | | | |
|---|---|---|---|---|---|---|
| Time Point | Initial | 1 M | 2 M | 3 M | 6 M | 9 M |
| Real Time (25° C./60% RH) | 113% | NA | NA | 108% | 105% | 111% |
| Accelerated (40° C./75% RH) | | 110% | 106% | 100% | 104% | NA |

N/A—testing not applicable

### Example 2

TABLE 6
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | calcium acetate | 0.2 | 17 | 25 |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Example 3

TABLE 7
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | zinc acetate | 0.2 | 17 | 25 |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Example 4

TABLE 8
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | a blend of magnesium acetate, | 0.2 | 17 | 30 |
| | iron acetate, copper acetate, | | | |
| | and manganese acetate | | | |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Example 5

TABLE 9
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | a blend of calcium acetate, | 0.2 | 17 | 30 |
| | copper acetate, manganese | | | |
| | acetate, and chromium acetate | | | |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Example 6

TABLE 10
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | a blend of zinc acetate, | 0.2 | 17 | 30 |
| | magnesium acetate, iron | | | |
| | acetate, copper acetate, | | | |
| | manganese acetate, and | | | |
| | chromium acetate | | | |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Example 7

TABLE 11
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | a blend of zinc acetate, | 0.2 | 17 | 30 |
| | magnesium acetate, manganese | | | |
| | acetate, and chromium acetate | | | |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Example 8

TABLE 10
capsule fill composition in liquid form

| | | | Example | |
|---|---|---|---|---|
| | | Minimum | approx. | Maximum |
| Ingredient | Example | % wt/wt | % wt/wt | % wt/wt |
| Apple cider vinegar | | 10 | 17 | 25 |
| powder (3-8% acetic | | | | |
| acid) | | | | |
| Mineral acetate | a blend of calcium acetate and | 0.2 | 17 | 30 |
| | copper acetate | | | |
| Secondary active | vitamin D3 | 0.01 | 0.03 | 10 |
| Edible Oile | extra virgin olive oil | 30 | 35 | 40 |
| Suspending agent | beeswax | 1 | 1.5 | 8 |
| Emulsifier/Wetting | sunflower lecithin | 1 | 1.5 | 8 |
| agent | | | | |

### Method of Treating a Subject in Need of an Acetic Acid Supplement

As used herein, a “subject” refers to a vertebrate, more particularly a mammal, for example, a human (or person) in need of a supplement to their diet, in particular, a supplement of acetic acid. The method of treating a person in need of an acetic acid supplement includes identifying such a person and administering one or more softgel capsule. In one embodiment, the individual softgel capsules contain a dose of 750 mg of acetic acid. In another embodiment, two, three, four, or five softgel capsules equals the dosage to administer 750 mg of acetic acid to the person. Numerous examples of softgel capsules with appropriate fill compositions were described herein and can be administered as a daily dose. The daily dose may be taken by the person for a period of days, weeks, months, or for the remainder of their life. Identifying the person in need of an acetic acid supplement may include medical professional testing or the recommendation of a medical professional.

The advantage of treatment with an acetic acid supplement as described herein may include appetite control, management of weight gain, maintenance of healthy cholesterol levels, and immune health support. The liquid softgel capsules are a great alternative to traditional liquid apple cider vinegar, each softgel is easy to transport and delivers all the benefits of liquid ACV without the tangy taste and odor.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the art will recognize and understand. For example, while processes can be presented in a given order, no order is to be implied in the claims unless expressly included in the claim. Each of these processes can be implemented in a variety of different ways, as those skilled in the art will appreciate. Also, while processes are at times shown as being performed in series, these processes can instead be performed in parallel, or can be performed, at different times. Further, any specific numbers noted herein are only examples—alternative implementations can employ differing values or ranges.

The teachings of the disclosure provided herein can be applied to other systems, not necessarily the system described above. The elements and acts of the various embodiments described above can be combined to provide further embodiments. Any patents noted above that are incorporated herein by reference, for example, can be modified, as necessary, to provide yet further embodiments of the disclosure provided herein. Further, while the above description describes certain embodiments, the teachings can be practiced in many ways that will be appreciated by those of skill in the art no matter how detailed the above appears in the text. Details of the capsule members, capsules, and related processes and products can vary considerably in their implementation details, while still being encompassed by the subject matter disclosed herein. Hence, although example embodiments of the invention have been shown and described, it is to be understood that all the terms used herein are descriptive rather than limiting and that many changes, modifications, and substitutions can be made by one having skill in the art without departing from the spirit and scope of the claims below.

1. A softgel capsule comprising:
a liquid fill composition comprising:
30% to 40% wt/wt of an edible oil or edible fat;
0.2% to 30% wt/wt of a mineral acetate powder, with the proviso that the mineral acetate powder does not comprise disodium acetate;
1% to 8% wt/wt of one or a combination of a suspending agent and/or a wetting agent; and
apple cider vinegar powder as a balance of the liquid fill material; and
an outer softgel shell encapsulating the liquid fill material.

2. The softgel capsule of claim 1, wherein the mineral acetate powder is a blend of calcium acetate powder and zinc acetate powder.

3. The softgel capsule of claim 2, wherein the zinc acetate powder comprises 3% to 6% wt/wt of the blend of calcium acetate powder and zinc acetate powder.

4. The softgel capsule of claim 2, wherein the zinc acetate powder comprises 4% to 5% wt/wt of the blend of calcium acetate powder and zinc acetate powder.

5. The softgel capsule of claim 1, further comprising a second active agent.

6. The softgel capsule of claim 5, wherein the second active agent is selected from the group consisting of oil soluble vitamins, water soluble vitamins, minerals, herbal extracts, amino acids, and combinations thereof.

7. The softgel capsule of claim 5, wherein the liquid fill material comprises 0.01% to 10% wt/wt of the second active agent.

8. The softgel capsule of claim 1, wherein a suspending agent and a wetting agent are present.

9. The softgel capsule of claim 8, wherein the suspending agent and wetting agent are present in a wt/wt ratio of 1:1.

10. The softgel capsule of claim 1, wherein the suspending agent is selected from the group consisting of beeswax, candelilla wax, carnauba wax, colloidal silicone dioxide, hydrophobic colloidal silica, hydrogenated vegetable oils, glycerol esters of fatty acids with low HLB values, polyvinylpyrrolidone, and mixtures of mono-, di-, and triglycerides/mono- and di-fatty acid esters of polyethylene glycols, gum Arabic, propylene glycol, alginate, Vitamin E polyethylene glycol succinate, and combinations thereof.

11. The softgel capsule of claim 1, wherein the wetting agent comprises lecithin.

12. The softgel capsule of claim 1, wherein the liquid fill composition comprises the edible oil selected from the group consisting of coconut oil, palm kernel oil, palm oil, soybean oil, safflower oil, cotton seed oil, rapeseed oil, poppy seed oil, corn oil, sunflower oil, olive oil, algae oil, fish oil, fish liver oil, seal oil, krill oil, squid oil, vegetable oil, and combinations thereof.

13. The softgel capsule of claim 1, wherein the liquid fill composition comprises the edible fat selected from the group consisting of lard, tallow fat, and combinations thereof.

14. The softgel capsule of claim 1, wherein the outer softgel shell has a composition comprising gelatin and a plasticizer or has a composition comprising a modified starch, carrageenan, and a plasticizer.

15. The softgel capsule of claim 1, wherein the fill composition comprises:
33% to 37% wt/wt of the edible oil or edible fat;
0.4% to 20% wt/wt of the mineral acetate powder; and
1% to 5% wt/wt of the combination of the suspending agent and the wetting agent.

16. The softgel capsule of claim 15, wherein the outer softgel shell has a composition comprising gelatin and a plasticizer or has a composition comprising a modified starch, carrageenan, and a plasticizer.

17. The softgel capsule of claim 15, further comprising a second active agent.

18. The softgel capsule of claim 17, wherein the liquid fill material comprises 0.01% to 10% wt/wt of the second active agent.

19. The softgel capsule of claim 15, wherein the mineral acetate powder is a blend of calcium acetate powder and zinc acetate powder.

20. The softgel capsule of claim 19, wherein the zinc acetate powder comprises 3% to 6% wt/wt of the blend of calcium acetate powder and zinc acetate powder.
